(19) United States
(12) Patent Application Publication
(10) Pub. No.: US 2025/0213608 A1
(43) Pub. Date: Jul. 3, 2025

(54) VANADYL SULFATE COMPOSITIONS AND METHODS OF USE THEREFOR FOR THE TREATEMENT AND PREVENTION OF AGING-RELATED DISEASES AND CANCER

(71) Applicant: The University of Chicago, Chicago, IL (US)

(72) Inventor: Jing CHEN, Chicago, IL (US)

(21) Appl. No.: 18/867,262

(22) PCT Filed: May 19, 2023
(86) PCT No.: PCT/US23/22850

§ 371 Date: Nov. 19, 2024

Related U.S. Application Data
(60) Provisional application No. 63/343,859, filed on May 19, 2022.

(51) Int. Cl. A61K 33/24; A61K 45/06; A61P 35/00; A61P 43/00

(52) U.S. Cl. CPC A61K 33/24; A61K 45/06; A61P 35/00; A61P 43/00

(57) ABSTRACT

Provided herein are pharmaceutical compositions and dietary supplements comprising vanadyl sulfate (VS) and method of use thereof for the induction of cell death in senescent cells. In particular. VS compositions find use in clearance of age-related and/or therapy-induced senescent cells and reduction/elimination of senescence associated secretory phenotype (SASP)-induced inflammatory environments in subjects. and therefore in the treatment/prevention of age-related conditions and/or cancer.

### CROSS-REFERENCE TO RELATED APPLICATIONS

This application claims the benefit of U.S. Provisional Patent Application No. 63/343,859, filed on May 19, 2022, which is incorporated by reference herein.

### FIELD

Provided herein are pharmaceutical compositions and dietary supplements comprising vanadyl sulfate (VS) and method of use thereof for the induction of cell death in senescent cells. In particular, VS compositions find use in clearance of age-related and/or therapy-induced senescent cells and reduction/elimination of senescence associated secretory phenotype (SASP)-induced inflammatory environments in subjects, and therefore in the treatment/prevention of age-related conditions and/or cancer.

### BACKGROUND

Senescence is characterized by cell-cycle arrest and secretion of various soluble and insoluble factors, termed the senescence associated secretory phenotype (SASP). Aberrant accumulation of senescent cells due to aging creates a SASP-induced inflammatory environment that leads to chronic tissue damage and contributes to diseases such as liver and lung fibrosis. In cancer treatment, therapy-induced senescence (TIS) by selective anti-cancer agents represents an effective cytostatic strategy to permanently abolish proliferative capacity of cancer cells without inducing cell death, leading to abrogated tumor growth or chronic tumors. However, senescent cancer cells persist and thus may escape senescent growth arrest to form more aggressive tumors, or produce SASP to promote immunosuppression or metastasis. Thus, senolytic drugs have been developed to selectively eliminate senescent cells to treat aging-related diseases, or to improve responses to anti-cancer therapy causing TIS. However, senolytic drugs generally suffer from in vivo “off-target” toxic side effects. What is needed are better-tolerated and effective senolytics for use in regimens for the treatment/prevention of aging-related diseases and/or cancer.

### SUMMARY

Provided herein are pharmaceutical compositions and dietary supplements comprising vanadyl sulfate (VS) and method of use thereof for the induction of cell death in senescent cells. In particular, VS compositions find use in clearance of age-related and/or therapy-induced senescent cells and reduction/elimination of senescence associated secretory phenotype (SASP)-induced inflammatory environments in subjects, and therefore in the treatment/prevention of age-related conditions and/or cancer.

In some embodiments, provided herein are methods of inducing cell death in senescent cells comprising administering vanadyl sulfate (VS) to a subject in need thereof. In some embodiments, the senescent cells are age-related senescent cells. In some embodiments, the senescent cells are therapy-induced senescent cells. In some embodiments, the subject suffers from senescence associated secretory phenotype (SASP). In some embodiments, the subject suffers from age-related dysfunctions caused by senescence. In some embodiments, the subject suffers from cardiovascular disease, a neurodegenerative disease, or a disease related to inflammation. In some embodiments, the lifespan of the subject is extended (e.g., the treatment extends the average lifespan of subjects in a population that are administered the treatment vs subjects not receiving the treatment).

In some embodiments, provided herein are methods of reducing treatment-associated side effects comprising co-administering vanadyl sulfate (VS) with a therapeutic or therapy for treating a disease. In some embodiments, the therapeutic or therapy results in induction of senescence in a population of cells. In some embodiments, the disease is cancer. In some embodiments, the therapeutic or therapy is selected from a chemotherapeutic, radiation, and an immunotherapeutic.

In some embodiments, provided herein are methods of treating cancer comprising co-administering vanadyl sulfate (VS) with a therapeutic or therapy for treating cancer to a subject suffering from cancer. In some embodiments, the therapeutic or therapy is selected from a chemotherapeutic, radiation, and an immunotherapeutic.

### BRIEF DESCRIPTION OF THE DRAWINGS

FIG. 1. Two-part screen of dietary substances with senolytic effects: (A) screen of compounds that exhibit a senolytic effect on cells exhibiting therapy-induced senescence; (B) screen of compounds that exhibit a senolytic effect on cells exhibiting p53 activation-induced senescence.

FIG. 2A-B. VS treatment selectively triggers cell death in senescent BJ cells (A) and HCA2 cells (B).

FIG. 3A-F. VS treatment ameliorates aging by eliminating senescent cells in vivo.

FIG. 4A-D. VS treatment shows therapeutic efficacy in bleomycin-induced lung fibrosis.

FIG. 5A-D. VS treatment shows therapeutic efficacy in CCl4-induced liver fibrosis.

FIG. 6A-B. VS suppresses tumor growth when combined with senescence induction in A549 xenograft model.

FIG. 7A-C. VS-induced cell death can be rescued by NAC.

FIG. 8A-B. Integrated KAS-seq, phosphor-antibody array and RNA-seq analyses.

FIG. 9A-B. VS treatment provides therapeutic efficacy in atherosclerosis model. Atherosclerosis-prone apolipoprotein E-deficient (ApoE^{-/-}) mice were used that display poor lipoprotein clearance with subsequent accumulation of cholesterol ester-enriched particles in the blood, which promote the development of atherosclerotic plaques. Compared to wild type control mice with no disease (WT-ND), the ApoE−/− mice developed atherosclerosis-like disease with accumulation of cholesterol that was stained with Sudan IV showing orange-red color (FIG. 9A, left). Quantitation results show that treatment with VS reduced accumulation of cholesterol in atherosclerotic plaques (FIG. 9A, right). In addition, VS reduced overall total cholesterol level in mice (FIG. 9B), indicating that VS could be used as both lipid-lowering and anti-atherosclerosis agent.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

The term “supplement” as used herein refers to a nutritional product that provides nutrients (e.g. vitamins, minerals, fatty acids) to a subject that may otherwise not be consumed in sufficient quantities (e.g., to enhance cancer treatment) by the subject. Supplements may be, for example, provided in the form of a pill, a tablet, a lozenge, a chewy capsule or tablet, a capsule, or a powder supplement that can be, for example, dissolved in water or a beverage (e.g., milk), or sprinkled on food. Supplements typically provide one or more selected compounds (e.g., VS) without providing a significant portion of the overall nutritional needs of a subject

The term “pharmaceutical formulation” as used herein refers to a composition comprising at least one pharmaceutically-active agent, chemical substance or drug. The pharmaceutical formulation may be in solid or liquid form and can comprise at least one additional active agent, carrier, vehicle, excipient or auxiliary agent identifiable by the skilled person. The pharmaceutical formulation may be in the form of a tablet, capsule, granules, powder, liquid or syrup.

The term “effective dose” or “effective amount” refers to an amount of an agent, e.g., a neutralizing antibody, that results in the reduction of symptoms in a patient, treatment of prevention of a disease or condition, or results in a desired biological outcome.

As used herein, the terms “administration” and “administering” refer to the act of giving a drug, prodrug, or other agent, or therapeutic to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “immunotherapy” refers to the treatment or prevention of a disease or condition by a method comprising inducing, enhancing, suppressing or otherwise modifying an immune response.

As used herein, the term “immunotherapeutic” refers to any agent (e.g., small molecule, peptide, antibody, engineered cell, etc.) capable of stimulating a host immune system to generate an immune response to a tumor or cancer in the subject.

### DETAILED DESCRIPTION

Provided herein are pharmaceutical compositions and dietary supplements comprising vanadyl sulfate (VS) and method of use thereof for the induction of cell death in senescent cells. In particular, VS compositions find use in clearance of age-related and/or therapy-induced senescent cells and reduction/elimination of senescence associated secretory phenotype (SASP)-induced inflammatory environments in subjects, and therefore in the treatment/prevention of age-related conditions and/or cancer.

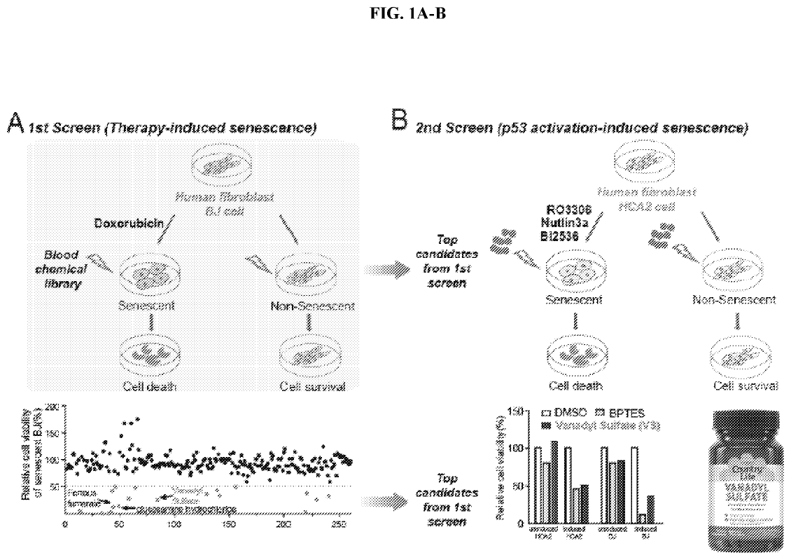

Experiments were conducted during development of embodiments herein to screen for diet-derived dietary substances, natural products, blood chemicals, etc. that provide senolytic activity with reduced “off-target” toxic side effects. A library of diet-derived blood chemicals was screened to identify compounds that selectively induce cell death in human fibroblast BJ cells with therapy-induced senescence by doxorubicin (FIG. 1A). The top candidates identified were tested in a second screen (FIG. 1B) using human fibroblast HCA2 cells with senescence induced by Nutlin3a (p53 activator), RO3306 (CDK1 inhibitor), and BI2436 (PLK1 inhibitor). The top candidate identified, vanadyl sulfate (VS), selectively induced cell death in senescent HCA2 and BJ cells but not in control uninduced cells.

VS is a mineral diet-supplement used by bodybuilders and athletes to boost muscle growth. VS is an oxidative form of vanadium that was suggested to “mimic” insulin signaling, which has been shown to reduce hyperglycemia and insulin resistance in animal models and improve hepatic and muscle insulin sensitivity in type 2 diabetes.

Experiments conducted during development of embodiments herein demonstrated that oral gavage of VS improved aging-related phenotypes in aged mice by reducing serum urea levels and gene expression levels of CDK inhibitor p16INK4a and inflammatory cytokine IL-6 in kidney tissues. In some embodiments, provided herein is the administration of VS as a pharmaceutical or nutraceutical composition to treat or prevent pathogenic senescence-related diseases (e.g., liver and lung fibrosis, atherosclerosis, diabetes, osteoarthritis, etc.). In some embodiments, provided herein is the administration of VS as a pharmaceutical or nutraceutical composition to treat or prevent various diverse human cancers, alone or in combination with anti-cancer therapies that result in therapy-induced senescence.

In some embodiments, VS is formulated for administration to a subject (e.g., human subject). In some embodiments, VS is formulated as a supplement. In some embodiments, provided herein is a supplement comprising of VS or and suitable carriers. In some embodiments, the supplement comprises VS and other nutrients, vitamins, and/or minerals. In some embodiments, the other components of a supplement are present to assist or enable delivery of VS or VS activity. In some embodiments, other components of a supplement are present to enhance the health of a subject or to otherwise effect the treatment (e.g., of cancer). Nutrients that may be provided with VS include, but are not limited to minerals (e.g., iron, manganese, magnesium, copper, calcium, phosphorous, etc.), vitamins (e.g., biotin, choline, folate, niacin, pantothenic acid, riboflavin, thiamin, and vitamins A, B6, B12, C, D, E, K, etc.), and other fatty acids (e.g., omega-3 fatty acids (e.g., α-linolenic acid (ALA), eicosapentaenoic acid (EPA), docosahexaenoic acid (DHA), etc.), omega-6 fatty acids (e.g., linoleic acid (LA), etc.), trans fatty acids, saturated fatty acids, unsaturated fatty acids, polyunsaturated fatty acids (PUFA), etc.). In some embodiments, a supplement comprises VS and one or more active bacterial cultures, wherein the bacterial cultures comprise species of bacteria that are beneficial to human health or the treatment/prevention of cancer. In some embodiments, a VS-containing supplement is formulated according to standard supplement formulations that are understood in the art. In some embodiments, VS is provided as a nutrient supplement to promote cell death of senescent cells.

In some embodiments, VS is formulated as a pharmaceutical composition. In some embodiments, VS and any co-formulated agents (when present) are provided in pharmaceutical formulations for administration to a subject by a suitable route. The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. Moreover, VS-containing pharmaceutical compositions are formulated into any suitable dosage form, including but not limited to, aqueous oral dispersions, liquids, gels, syrups, elixirs, slurries, suspensions, aerosols, fast melt formulations, effervescent formulations, lyophilized formulations, tablets, powders, pills, dragees, and capsules.

Pharmaceutical preparations comprising VS provided for oral use can be obtained by mixing one or more solid excipients with VS (and other therapeutic agents desired in the formulation) with any suitable substituents and functional groups disclosed herein, optionally grinding the resulting mixture, and processing the mixture of granules, after adding suitable auxiliaries, if desired, to obtain tablets, pills, or capsules. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

In some embodiments, routes of administration, formation of VS, etc. are selected to provide efficient and effective delivery. In some embodiments, VS is provided with a suitable carrier. In some embodiments, VS is encapsulated of embedded into a carrier. In some embodiments, a carrier may comprise a liposome, nanoparticle, or other suitable system for delivery of VS. In some embodiments, VS is conjugated to a carrier molecule. Suitable carrier molecules for conjugation of VS may include small molecules, peptides, proteins, polymers, etc. In some embodiments, the carrier and/or delivery system is selected to optimize the solubility, stability, bioavailability, targeting, etc. of the VS.

The supplement or pharmaceutical compositions described herein may be in unit dosage forms suitable for single administration of precise dosages. In unit dosage form, the formulation is divided into unit doses containing appropriate quantities of VS. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition.

Dosing and administration regimes are tailored by the clinician, or others skilled in supplements or the pharmacological arts, based upon well-known pharmacological and therapeutic considerations including, but not limited to, the desired level of therapeutic effect, and the practical level of therapeutic effect obtainable. Generally, it is advisable to follow well-known pharmacological principles for administrating chemotherapeutic agents (e.g., it is generally advisable to not change dosages by more than 50% at time and no more than every 3-4 agent half-lives). For compositions that have relatively little or no dose-related toxicity considerations, and where maximum efficacy is desired, doses in excess of the average required dose are not uncommon. This approach to dosing is commonly referred to as the “maximal dose” strategy. In certain embodiments, VS is administered to a subject at a dose of about 0.01 mg/kg to about 200 mg/kg (e.g., 0.01 mg/kg, 0.02 mg/kg, 0.05 mg/kg, 0.1 mg/kg, 0.2 mg/kg, 0.5 mg/kg, 1 mg/kg, 2 mg/kg, 5 mg/kg, 10 mg/kg, 20 mg/kg, 50 mg/kg, 100 mg/kg, 200 mg/kg, or ranges therebetween). Dosing may be once per day, multiple times per day (e.g., 2, 3, 4, etc.), once per week, or according to any suitable protocol. In some embodiments, VS is administered a single time, each time a co-administered agent is delivered, or for a time period of days, weeks, months, indefinitely, etc.

In some embodiments, VS is administered (e.g., as a pharmaceutical, supplement, nutraceutical, etc.) to a subject suffering from or at risk of a disease or condition related to senescence. In some embodiments, the senescence is therapy-induced senescence and the VS is administered to reduce/eliminate the damaging effects of the senescence that is induced in the treatment of another condition/disease (e.g., cancer). In other embodiments, VS is administered to treat or prevent diseases or conditions that results from senescence that occurs naturally (e.g., age-related, as part of a disease process, etc.) in the subject.

Some embodiments herein are directed to administration of VS (e.g., as a pharmaceutical, supplement, nutraceutical, etc.) to a subject for the treatment, prevention, or symptom-reduction of diseases and/or conditions in which cellular senescence (and/or the accumulation of senescent cells) has a causal and/or contributory role, such as osteoporosis, frailty, cardiovascular diseases (atherosclerosis, congestive heart failure, etc.), osteoarthritis, pulmonary fibrosis, renal diseases, neurodegenerative diseases (e.g., Alzheimer's disease, Parkinson's disease, etc.), hepatic steatosis, and metabolic dysfunction (e.g. diabetes, NAFLD, metabolic syndrome, etc.), neuromuscular dysfunction (e.g. sarcopenia, osteoporosis, NMJ stability, etc.), etc

In some embodiments, methods herein are provided for the treatment/prevention/symptom reduction of any senescence-related diseases. Examples of the senescence-related diseases include Alzheimer's disease, Parkinson's disease, cataract, macular degeneration, glaucoma, atherosclerosis, acute coronary syndrome, myocardial infarction, stroke, hypertension, idiopathic pulmonary fibrosis (IPF), chronic obstructive pulmonary disease (COPD), osteoarthritis, coronary artery disease, cerebrovascular disease, periodontal disease, atrophy or fibrosis in various tissues, brain or heart injury, treatment-related myelodysplastic syndrome, and the like. The senescence-related diseases may also include Hutchinson-Gilford progeria syndrome, Werner syndrome, Cockayne syndrome, xeroderma pigmentosum, ataxia telangiectasia, Fanconi anemia, neuropathic anemia, and the like.

Further examples of the senescence-related diseases include circulatory diseases, such as cardiovascular diseases, e.g. angina pectoris, aortic aneurysms, arrhythmia, cerebral aneurysms, diastolic dysfunction, cardiac fibrosis, cardiomyopathy, carotid artery disease, coronary thrombosis, endocarditis, hypercholesterolemia, hyperlipidemia, mitral valve prolapse, and peripheral vascular disease; inflammatory or autoimmune diseases, such as disc herniation, oral mucositis, erythema, interstitial cystitis, scleroderma, and alopecia; neurodegenerative diseases, such as dementia, Huntington's disease, motor neuron dysfunction, memory loss associated with aging, depression, and mood disorder; metabolic disease such as metabolic syndrome; pulmonary diseases, such as decreased pulmonary function associated with aging, asthma, bronchiectasis, cystic fibrosis, and emphysema; gastrointestinal diseases such as Barrett's esophagus; diseases associated with aging, such as hepatic fibrosis, muscle fatigue, oral mucosa fibrosis, pancreatic fibrosis, benign prostatic hyperplasia (BPH), and sleep disorder; reproductive failure, such as climacterium, decreased ovum supply, decreased sperm viability, decreased fertility, decreased libido, decreased erection, and excitation; skin diseases, such as atopic dermatitis, skin erythema, cutaneous lymphoma, dysesthesia, eczema, eosinophilic dermatitis, fibrotic proliferation of skin, hyperpigmentation, immuno blistering disease, nevus, pemphigus vulgaris, itching, psoriasis, rash, reactive neutrophilic skin disease, wrinkles, and urticaria; posttransplant kidney fibrosis; carotid artery thrombosis; and the like.

Specific and non-limiting examples of senescence-related diseases that may be treated in embodiments herein include circulatory diseases, such as heart failure, atherosclerosis, arteriosclerotic cerebrovascular or cardiovascular disease, and hypertension; cerebrovascular diseases, such as cerebral infarction and cerebral hemorrhage; metabolic diseases such as dyslipidemia; respiratory diseases, such as pulmonary fibrosis and emphysema; locomotive syndromes, such as skeletal muscle atrophy (sarcopenia) and osteoarthritis; geriatric syndromes, such as dementia and frailty; cancer; chronic kidney disease; ocular diseases, such as cataract, glaucoma, age-related macular degeneration, and presbyopia; age-related alopecia; age-related hearing loss; pain associated with aging, such as lumbar pain and joint pain; skin diseases, such as asteatotic eczema and cutaneous pruritus; liver diseases, such as fatty liver, nonalcoholic steatohepatitis (NASH), and liver cirrhosis; bone diseases, such as osteoporosis and osteoarthropathy; premature aging, such as Hutchinson-Gilford progeria syndrome, Werner syndrome, Cockayne syndrome, and Rothmund-Thomson syndrome; and the like. In some embodiments, VS is administered to prevent aging and/or effects thereof.

Certain embodiments herein are directed to administration of VS (e.g., as a pharmaceutical, supplement, nutraceutical, etc.) to a subject with cancer, in remission from cancer, or at elevated risk of cancer. In some embodiments, VS is administered as part of therapeutic or prophylactic regimen for the treatment or prevention of acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer.

The induction of tumor cell senescence has been well-established as a prominent therapeutic response of cancer cells to chemotherapy, radiation, small-molecule inhibitor-based targeted therapeutics and immunotherapy (Qin et al. World J Clin Oncol. 2018 Dec. 20; 9 (8): 180-187.; incorporated by reference in its entirety). In some embodiments, VS is co-administered (e.g., before, during, and/or after administration of the co-therapy) with one or more cancer therapies to prevent/treat/reduce/eliminate senescent cells, senescence associated secretory phenotype (SASP), SASP-induced inflammatory environments, and/or to enhance cancer treatment.

Particularly, when VS is used as part of a cancer treatment, VS may be co-administered with one or more chemotherapeutics. Many chemotherapeutics are presently known in the art and can be used in combination with VS. In some embodiments, the chemotherapeutic is selected from the group consisting of mitotic inhibitors, alkylating agents, anti-metabolites, intercalating antibiotics, growth factor inhibitors, cell cycle inhibitors, enzyme inhibitors, topoisomerase inhibitors, protein-protein interaction inhibitors, biological response modifiers, anti-hormones, angiogenesis inhibitors, and anti-androgens.

Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin: phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”): cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4 (5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin: chlorambucil; gemcitabine; 6-thioguanine: mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). Where desired, the compounds or pharmaceutical composition of the present invention can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris (2-chloroethyl) amine, Troxacitabine, Uramustine, Vadimezan, Vinflunine, ZD6126 or Zosuquidar.

Embodiments herein further relate to methods for using VS in combination with radiation therapy for inhibiting abnormal cell growth or treating the hyperproliferative disorder in the mammal. Techniques for administering radiation therapy are known in the art, and these techniques can be used in the combination therapy described herein. The administration of VS in this combination therapy can be determined as described herein. Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

VS may also be used in combination with an amount of one or more substances selected from anti-angiogenesis agents, signal transduction inhibitors, antiproliferative agents, glycolysis inhibitors, or autophagy inhibitors.

Anti-angiogenesis agents, such as MMP-2 (matrix-metalloproteinasc 2) inhibitors, MMP-9 (matrix-metalloprotienase 9) inhibitors, and COX-11 (cyclooxygenase 11) inhibitors, can be used in conjunction with VS. Anti-angiogenesis agents include, for example, rapamycin, temsirolimus (CCI-779), everolimus (RAD001), sorafenib, sunitinib, and bevacizumab. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), United States Patent 5,863, 949 (issued Jan. 26, 1999), United States Patent 5,861, 510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or AMP-9 relative to the other matrix-metalloproteinases (e.g., MAP-1, MMP-3, MMP-4, MMP-5, MMP-6, MMP-7, MMP-8, MMP-10, MMP-11, MMP-12, andMMP-13). Some specific examples of MMP inhibitors useful in the invention are AG-3340, RO 32-3555, and RS 13-0830.

Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used.

In some embodiments, medicaments which are administered in conjunction with VS include any suitable drugs usefully delivered by inhalation for example, analgesics, e.g., codeine, dihydromorphine, ergotamine, fentanyl or morphine; anginal preparations, e.g., diltiazem; antiallergics, e.g., cromoglycate, ketotifen or nedocromil; anti-infectives, e.g., cephalosporins, penicillins, streptomycin, sulphonamides, tetracyclines or pentamidine; antihistamines, e.g., methapyrilene; anti-inflammatoires, e.g., beclomethasone, flunisolide, budesonide, tipredane, triamcinolone acetonide or fluticasone; antitussives, e.g., noscapine; bronchodilators, e.g., ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g., amiloride; anticholinergics e.g., ipratropium, atropine or oxitropium; hormones, e.g., cortisone, hydrocortisone or prednisolone; xanthines e.g., aminophylline, choline theophyllinate, lysine theophyllinate or theophylline; and therapeutic proteins and peptides, e.g., insulin or glucagon

Exemplary therapeutic agents useful for a combination therapy with VS include but are not limited to agents as described above, radiation therapy, hormone antagonists, hormones and their releasing factors, thyroid and antithyroid drugs, estrogens and progestins, androgens, adrenocorticotropic hormone; adrenocortical steroids and their synthetic analogs; inhibitors of the synthesis and actions of adrenocortical hormones, insulin, oral hypoglycemic agents, and the pharmacology of the endocrine pancreas, agents affecting calcification and bone turnover: calcium, phosphate, parathyroid hormone, vitamin D, calcitonin, vitamins such as water-soluble vitamins, vitamin B complex, ascorbic acid, fat-soluble vitamins, vitamins A, K, and E, growth factors, cytokines, chemokines, muscarinic receptor agonists and antagonists; anticholinesterase agents; agents acting at the neuromuscular junction and/or autonomic ganglia; catecholamines, sympathomimetic drugs, and adrenergic receptor agonists or antagonists; and 5-hydroxytryptamine (5-HT, serotonin) receptor agonists and antagonists.

Other suitable therapeutic agents for coadministration with VS also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, cicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, β-adrenergic agonists, ipratropium, glucocorticoids, methylxanthines, sodium channel blockers, opioid receptor agonists, calcium channel blockers, membrane stabilizers and leukotriene inhibitors.

Additional therapeutic agents contemplated for co-administration with VS include diuretics, vasopressin, agents affecting the renal conservation of water, rennin, angiotensin, agents useful in the treatment of myocardial ischemia, anti-hypertensive agents, angiotensin converting enzyme inhibitors, β-adrenergic receptor antagonists, agents for the treatment of hypercholesterolemia, and agents for the treatment of dyslipidemia.

Other therapeutic agents contemplated for co-administration with VS include drugs used for control of gastric acidity, agents for the treatment of peptic ulcers, agents for the treatment of gastroesophageal reflux disease, prokinetic agents, antiemetics, agents used in irritable bowel syndrome, agents used for diarrhea, agents used for constipation, agents used for inflammatory bowel disease, agents used for biliary disease, agents used for pancreatic disease. Therapeutic agents used to treat protozoan infections, drugs used to treat Malaria, Amebiasis, Giardiasis, Trichomoniasis, Trypanosomiasis, and/or Leishmaniasis, and/or drugs used in the chemotherapy of helminthiasis. Other therapeutic agents include antimicrobial agents, sulfonamides, trimethoprim-sulfamethoxazole quinolones, and agents for urinary tract infections, penicillins, cephalosporins, and other, β-lactam antibiotics, an agent comprising an aminoglycoside, protein synthesis inhibitors, drugs used in the chemotherapy of tuberculosis, mycobacterium avium complex disease, and leprosy, antifungal agents, antiviral agents including nonretroviral agents and antiretroviral agents.

Examples of therapeutic antibodies that can be combined with VS include but are not limited to anti-receptor tyrosine kinase antibodies (cetuximab, panitumumab, trastuzumab), anti CD20 antibodies (rituximab, tositumomab), and other antibodies such as alemtuzumab, bevacizumab, and gemtuzumab.

Moreover, therapeutic agents used for immunomodulation, such as immunomodulators, immunosuppressive agents, tolerogens, and immunostimulants are contemplated by the methods herein. In addition, therapeutic agents acting on the blood and the blood-forming organs, hematopoietic agents, growth factors, minerals, and vitamins, anticoagulant, thrombolytic, and antiplatelet drugs.

For treating renal carcinoma, one may combine VS with sorafenib and/or avastin. For treating an endometrial disorder, one may combine VS with doxorubincin, taxotere (taxol), and/or cisplatin (carboplatin). For treating ovarian cancer, one may combine VS with cisplatin (carboplatin), taxotere, doxorubincin, topotecan, and/or tamoxifen. For treating breast cancer, one may combine VS with taxotere (taxol), gemcitabine (capecitabine), tamoxifen, letrozole, tarceva, lapatinib, PD0325901, avastin, herceptin, OSI-906, and/or OSI-930. For treating lung cancer, one may combine a compound of the present invention with taxotere (taxol), gemcitabine, cisplatin, pemetrexed, Tarceva, PD0325901, and/or avastin.

Further therapeutic agents that can be combined with a compound herein are found in Goodman and Gilman's “The Pharmacological Basis of Therapeutics” Tenth Edition edited by Hardman, Limbird and Gilman or the Physician's Desk Reference, both of which are incorporated herein by reference in their entirety.

In some embodiments, VS is co-administered with another therapeutic agent effective in treating leukemia and/or other cancers. In some embodiments, VS is co-administered with one or more therapeutic agents approved for the treatment of Acute Lymphoblastic Leukemia (ALL), for example: ABITREXATE (Methotrexate), ADRIAMYCIN PFS (Doxorubicin Hydrochloride), ADRIAMYCIN RDF (Doxorubicin Hydrochloride), ARRANON (Nelarabine), Asparaginase Erwinia chrysanthemi, CERUBIDINE (Daunorubicin Hydrochloride), CLAFEN (Cyclophosphamide), CLOFARABINE, CLOFAREX (Clofarabine), CLOLAR (Clofarabine), Cyclophosphamide, Cytarabine, CYTOSAR-U (Cytarabine), CYTOXAN (Cyclophosphamide), Dasatinib, Daunorubicin Hydrochloride, Doxorubicin Hydrochloride, Erwinaze (Asparaginase Erwinia Chrysanthemi), FOLEX (Methotrexate), FOLEX PFS (Methotrexate), GLEEVEC (Imatinib Mesylate), ICLUSIG (Ponatinib Hydrochloride), Imatinib Mesylate, MARQIBO (Vincristine Sulfate Liposome), Methotrexate, METHOTREXATE LPF (Methorexate), MEXATE (Methotrexate), MEXATE-AQ (Methotrexate), Nelarabine, NEOSAR (Cyclophosphamide), ONCASPAR (Pegaspargase), Pegaspargase, Ponatinib Hydrochloride, RUBIDOMYCIN (Daunorubicin Hydrochloride), SPRYCEL (Dasatinib), TARABINE PFS (Cytarabine), VINCASAR PFS (Vincristine Sulfate), Vincristine Sulfate, etc.

In some embodiments, VS is co-administered with one or more therapeutic agents approved for the treatment of Acute Myeloid Leukemia (AML), for example: ADRIAMYCIN PFS (Doxorubicin Hydrochloride), ADRIAMYCIN RDF (Doxorubicin Hydrochloride), Arsenic Trioxide, CERUBIDINE (Daunorubicin Hydrochloride), CLAFEN (Cyclophosphamide), Cyclophosphamide, Cytarabine, CYTOSAR-U (Cytarabine), CYTOXAN (Cyclophosphamide), Daunorubicin Hydrochloride, Doxorubicin Hydrochloride, NEOSAR (Cyclophosphamide), RUBIDOMYCIN (Daunorubicin Hydrochloride), RYDAPT (Midostaurin), TARABINE PFS (Cytarabine), TRISENOX (Arsenic Trioxide), VINCASAR PFS (Vincristine Sulfate), Vincristine Sulfate, etc.

In some embodiments, VS is co-administered with one or more therapeutic agents approved for the treatment of Chronic Lymphocytic Leukemia (CLL), for example: Alemtuzumab, AMBOCHLORIN (Chlorambucil), AMBOCLORIN (Chlorambucil), ARZERRA (Ofatumumab), Bendamustine Hydrochloride, CAMPATH (Alemtuzumab), CHLORAMBUCILCLAFEN (Cyclophosphamide), Cyclophosphamide, CYTOXAN (Cyclophosphamide), FLUDARA (Fludarabine Phosphate), Fludarabine Phosphate, LEUKERAN (Chlorambucil), LINFOLIZIN (Chlorambucil), NEOSAR (Cyclophosphamide), Ofatumumab, TREANDA (Bendamustine Hydrochloride), etc.

In some embodiments, VS is co-administered with one or more therapeutic agents approved for the treatment of Chronic Myelogenous Leukemia (CML), for example: BOSULIF (Bosutinib), Bosutinib, CLAFEN (Cyclophosphamide), Cyclophosphamide, Cytarabine, CYTOSAR-U (Cytarabine), CYTOXAN (Cyclophosphamide), Dasatinib, GLEEVEC (Imatinib Mesylate), ICLUSIG (Ponatinib Hydrochloride), Imatinib Mesylate, NEOSAR (Cyclophosphamide), Nilotinib, Omacetaxine Mepesuccinate, Ponatinib Hydrochloride, SPRYCEL (Dasatinib), SYNRIBO (Omacetaxine Mepesuccinate), TARABINE PFS (Cytarabine), TASIGNA (Nilotinib), etc.

In some embodiments, VS is co-administered with one or more therapeutic agents approved for the treatment of Meningeal Leukemia, for example: CYTARABINE, CYTOSAR-U (Cytarabine), TARABINE PFS (Cytarabine), etc.

In some embodiments, VS is co-administered with one or more alkylating agents (e.g., for the treatment of cancer) selected from, for example, nitrogen mustard N-oxide, cyclophosphamide, ifosfamide, thiotepa, ranimustine, nimustine, temozolomide, altretamine, apaziquone, brostallicin, bendamustine, carmustine, estramustine, fotemustine, glufosfamide, mafosfamide, bendamustin, mitolactol, cisplatin, carboplatin, eptaplatin, lobaplatin, nedaplatin, oxaliplatin, and satraplatin.

In some embodiments, VS is co-administered with one or more anti-metabolites (e.g., for the treatment of cancer) selected from, for example, methotrexate, 6-mercaptopurineriboside, mercaptopurine, 5-fluorouracil, tegafur, doxifluridine, carmofur, cytarabine, cytarabine ocfosfate, enocitabine, gemcitabine, fludarabin, 5-azacitidine, capecitabine, cladribine, clofarabine, decitabine, eflornithine, ethynylcytidine, cytosine arabinoside, hydroxyurea, melphalan, nelarabine, nolatrexed, ocfosf[iota]te, disodium premetrexed, pentostatin, pelitrexol, raltitrexed, triapine, trimetrexate, vidarabine, vincristine, and vinorelbine;

In some embodiments, VS is co-administered with one or more hormonal therapy agents (e.g., for the treatment of cancer) selected from, for example, exemestane, Lupron, anastrozole, doxercalciferol, fadrozole, formestane, abiraterone acetate, finasteride, epristeride, tamoxifen citrate, fulvestrant, Trelstar, toremifene, raloxifene, lasofoxifene, letrozole, sagopilone, ixabepilone, epothilone B, vinblastine, vinflunine, docetaxel, and paclitaxel;

In some embodiments, VS is co-administered with one or more cytotoxic topoisomerase inhibiting agents (e.g., for the treatment of cancer) selected from, for example, aclarubicin, doxorubicin, amonafide, belotecan, camptothecin, 10-hydroxycamptothecin, 9-aminocamptothecin, diflomotecan, irinotecan, topotecan, edotecarin, epimbicin, etoposide, exatecan, gimatecan, lurtotecan, mitoxantrone, pirambicin, pixantrone, rubitecan, sobuzoxane, tafluposide, etc.

In some embodiments, VS is co-administered with one or more anti-angiogenic compounds (e.g., for the treatment of cancer) selected from, for example, acitretin, aflibercept, angiostatin, aplidine, asentar, axitinib, recentin, bevacizumab, brivanib alaninat, cilengtide, combretastatin, DAST, endostatin, fenretinide, halofuginone, pazopanib, ranibizumab, rebimastat, removab, revlimid, sorafenib, vatalanib, squalamine, sunitinib, telatinib, thalidomide, ukrain, and vitaxin.

In some embodiments, VS is co-administered with one or more antibodies (e.g., for the treatment of cancer) selected from, for example, trastuzumab, cetuximab, bevacizumab, rituximab, ticilimumab, ipilimumab, lumiliximab, catumaxomab, atacicept, oregovomab, and alemtuzumab.

In some embodiments VS is co-administered with one or more VEGF inhibitors (e.g., for the treatment of cancer) selected from, for example, sorafenib, DAST, bevacizumab, sunitinib, recentin, axitinib, aflibercept, telatinib, brivanib alaninate, vatalanib, pazopanib, and ranibizumab.

In some embodiments, VS is co-administered with one or more EGFR inhibitors (e.g., for the treatment of cancer) selected from, for example, cetuximab, panitumumab, vectibix, gefitinib, erlotinib, and Zactima.

In some embodiments, VS is co-administered with one or more HER2 inhibitors (e.g., for the treatment of cancer) selected from, for example, lapatinib, tratuzumab, and pertuzumab; CDK inhibitor is selected from roscovitine and flavopiridol;

In some embodiments, VS is co-administered with one or more proteasome inhibitors (e.g., for the treatment of cancer) selected from, for example, bortezomib and carfilzomib.

In some embodiments, VS is co-administered with one or more serine/threonine kinase inhibitors (e.g., for the treatment of cancer), for example, MEK inhibitors and Raf inhibitors such as sorafenib.

In some embodiments, VS is co-administered with one or more tyrosine kinase inhibitors (e.g., for the treatment of cancer) selected from, for example, dasatinib, nilotibib, DAST, bosutinib, sorafenib, bevacizumab, sunitinib, AZD2171, axitinib, aflibercept, telatinib, imatinib mesylate, brivanib alaninate, pazopanib, ranibizumab, vatalanib, cetuximab, panitumumab, vectibix, gefitinib, erlotinib, lapatinib, tratuzumab, pertuzumab and midostaurin

In some embodiments, VS is co-administered with one or more androgen receptor antagonists (e.g., for the treatment of cancer) selected from, for example, nandrolone decanoate, fluoxymesterone, Android, Prostaid, andromustine, bicalutamide, flutamide, apocyproterone, apoflutamide, chlormadinone acetate, Androcur, Tabi, cyproterone acetate, and nilutamide.

In some embodiments, a compound described herein is co-administered with one or more aromatase inhibitors (e.g., for the treatment of cancer) selected from, for example, anastrozole, letrozole, testolactone, exemestane, aminoglutethimide, and formestane.

In some embodiments, VS is co-administered with one or more other anti-cancer agents including, e.g., alitretinoin, ampligen, atrasentan bexarotene, borte-zomib, bosentan, calcitriol, exisulind, fotemustine, ibandronic acid, miltefosine, mitoxantrone, 1-asparaginase, procarbazine, dacarbazine, hydroxycarbamide, pegaspargase, pentostatin, tazaroten, velcade, gallium nitrate, canfosfamide, darinaparsin, and tretinoin. In a preferred embodiment, the compounds of the present disclosure may be used in combination with chemotherapy (e.g., cytotoxic agents), anti-hormones and/or targeted therapies such as other kinase inhibitors, mTOR inhibitors and angiogenesis inhibitors.

In some embodiments, VS is co-administered with one or more immunotherapeutics. In some embodiments, VS is co-administered with a T-cell-based immunotherapeutic. some embodiments, VS is co-administered with one or more immunotherapeutics selected from a therapy comprising the administration of immune checkpoint inhibitor, CAR-T cell therapy, monoclonal antibody therapy, and bispecific T-cell engager therapy. In some embodiments, VS is co-administered with an immune checkpoint inhibitor that binds to and inhibits the activity of an immune checkpoint protein is selected from the group consisting of CTLA4, PD-1, PD-L1, PD-L2, A2AR, B7-H3, B7-H4, BTLA, KIR, LAG3, TIM-3 or VISTA. In some embodiments, the VS is co-administered with an immune checkpoint inhibitor selected from the group consisting of nivolumab, pembrolizumab, pidilizumab, AMP-224, AMP-514, STI-A1110, TSR-042, RG-7446, BMS-936559, BMS-936558, MK-3475, MPDL3280A, MEDI-4736, MSB-0020718C, AUR-012 and STI-A1010.

VS may be used in combination with the agents disclosed herein or other suitable agents, depending on the condition being treated. When used in a combination therapy, VS is administered simultaneously or separately with the second agent. This administration in combination can include simultaneous administration of the two agents in the same dosage form, simultaneous administration in separate dosage forms, and separate administration. That is, VS and any of the agents described above can be formulated together in the same dosage form and administered simultaneously. Alternatively, VS and any of the agents described above can be simultaneously administered, wherein both the agents are present in separate formulations. In another alternative, VS can be administered just followed by and any of the agents described above, or vice versa. In some embodiments of the separate administration protocol, VS and any of the agents described above are administered a few minutes apart, or a few hours apart, or a few days apart.

In embodiments in which the VS used for the treatment or prevention of non-cancer diseases and/or conditions, VS may be co-administered with therapeutics and/or therapies known in the field to be appropriate for the treatment of such diseases and/or conditions.

In some embodiments, provided herein are compositions (e.g., pharmaceutical compositions, nutraceuticals, foods, supplements, etc.) comprising VS. In some embodiments, provided herein are compositions (e.g., pharmaceutical compositions, nutraceuticals, foods, supplements, etc.) comprising VS and one or more additional agents (e.g., the agents described above) for the treatment of cancer or an aging-related condition (e.g., fibrosis (e.g., liver fibrosis, lung fibrosis, etc.).

### Experimental

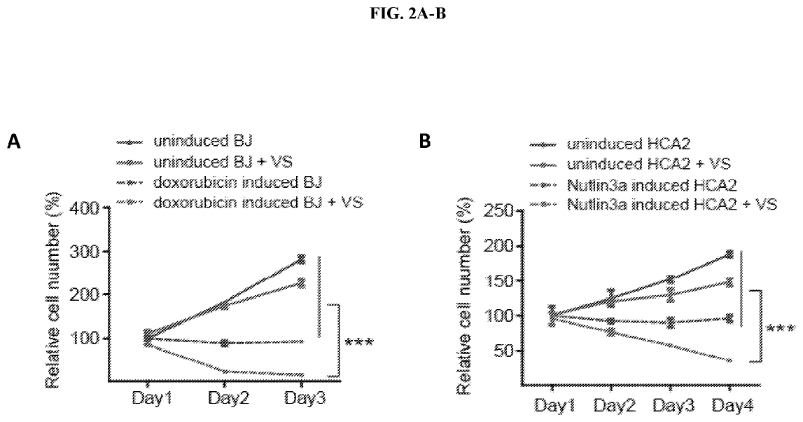

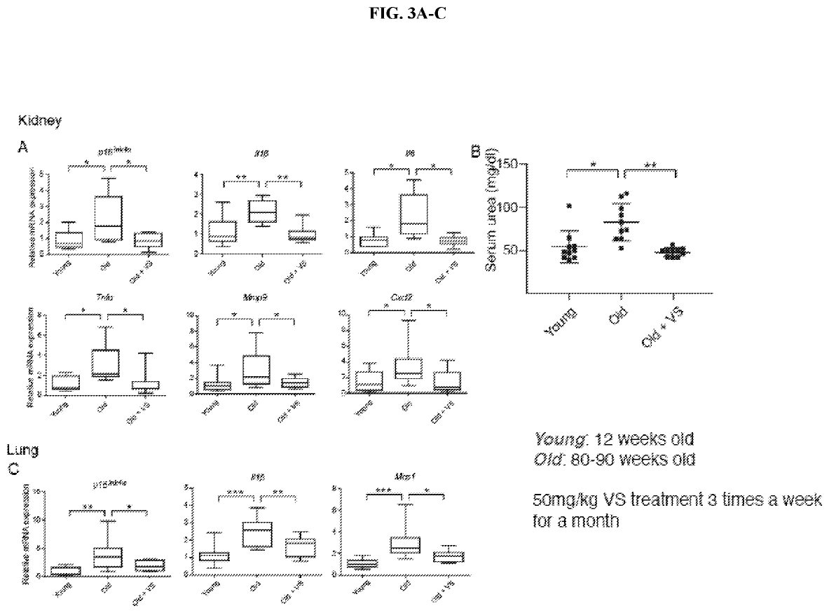

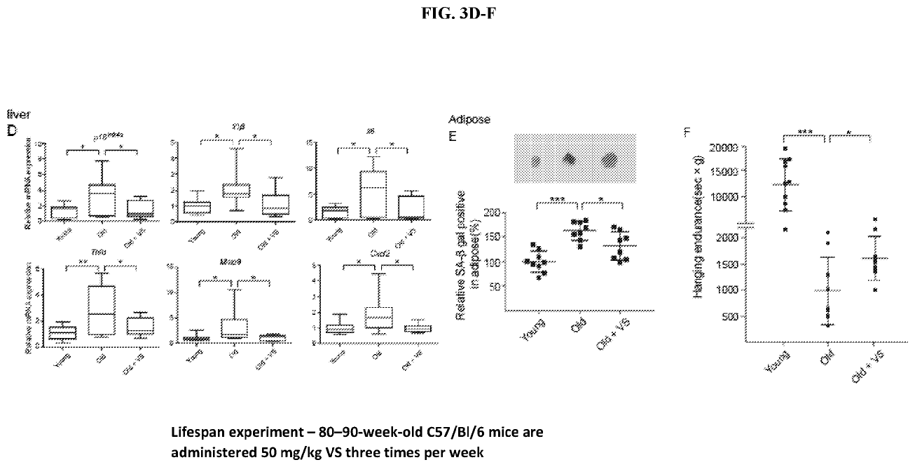

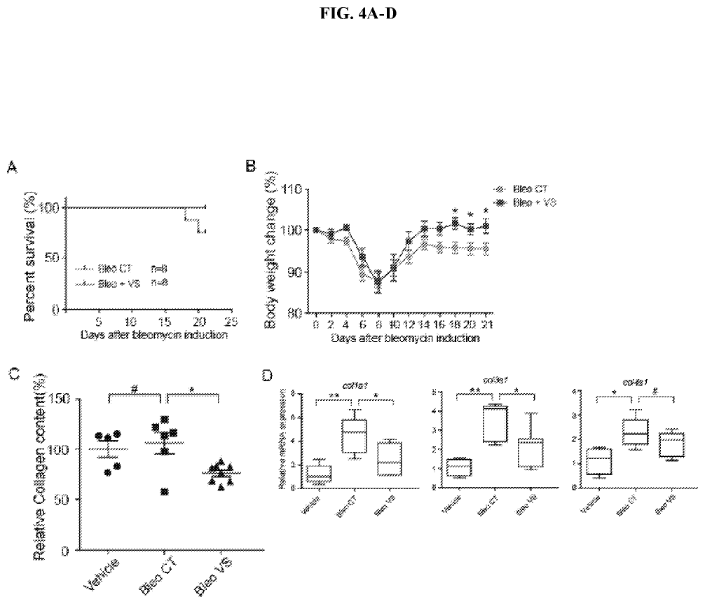

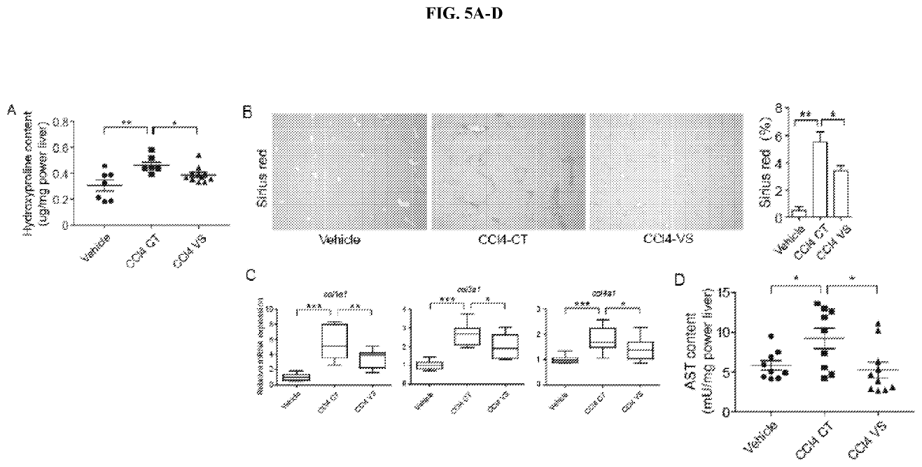

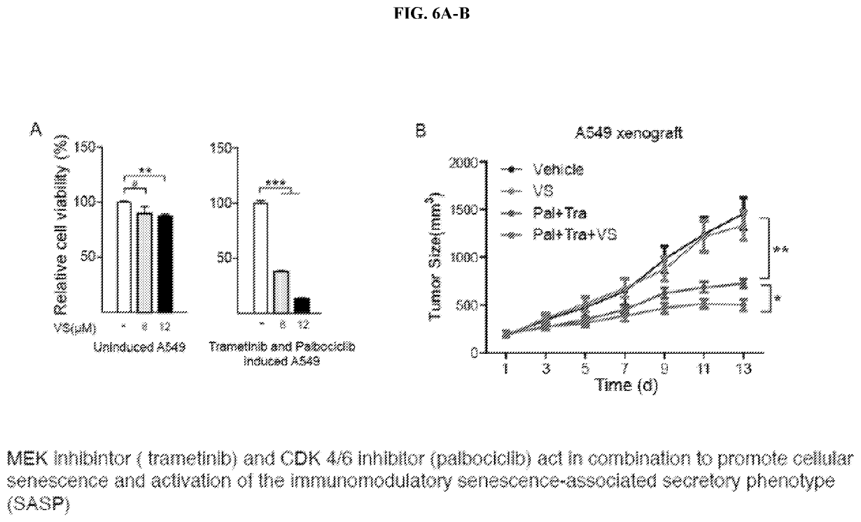

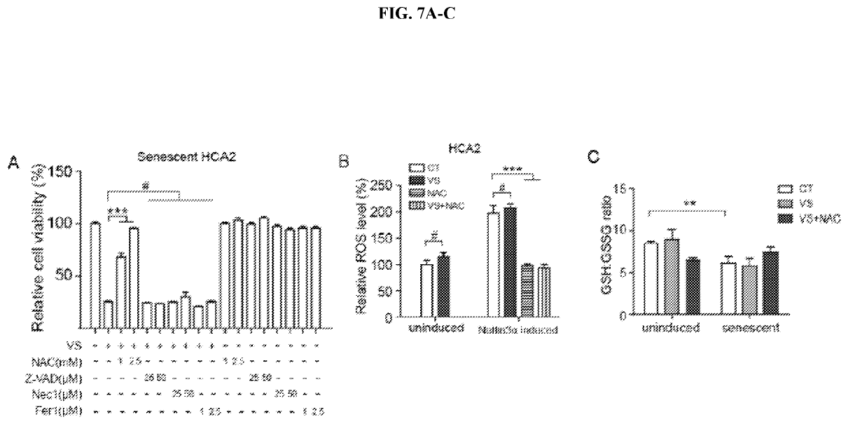

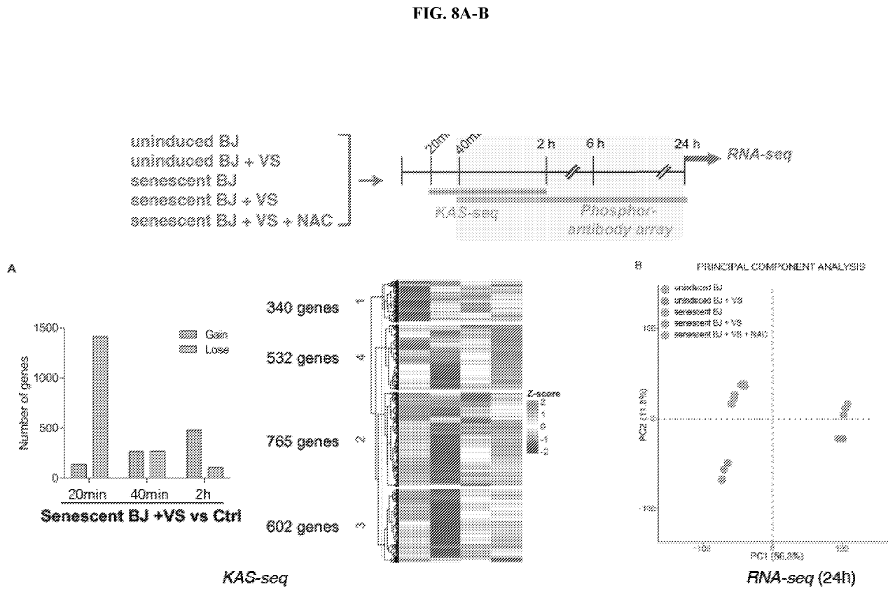

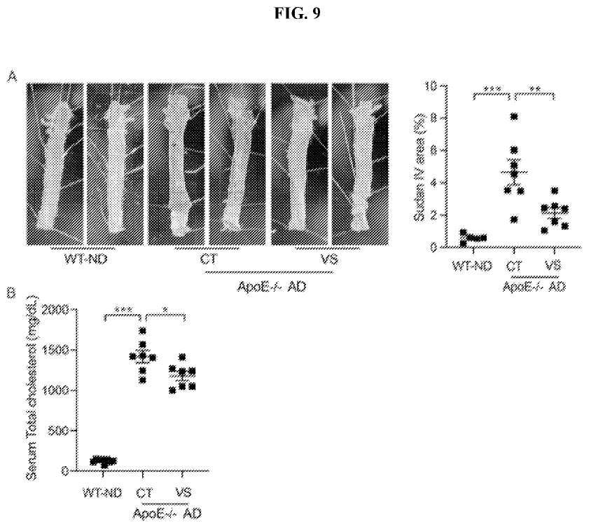

Experiments were conducted during development of embodiments herein to screen for natural products that provide senolytic activity. A library of diet-derived blood chemicals was screened to identify compounds that selectively induce cell death in human fibroblast BJ cells with therapy-induced senescence by doxorubicin (FIG. 1A). The top candidates identified were tested in a second screen (FIG. 1B) using human fibroblast HCA2 cells with senescence induced by Nutlin3a (p53 activator), RO3306 (CDK1 inhibitor), and BI2436 (PLK1 inhibitor). The top candidate identified, vanadyl sulfate (VS), selectively induced cell death in senescent HCA2 and BJ cells but not in control uninduced cells. Additional experiment demonstrated that: VS treatment selectively triggers cell death in senescent cells (FIG. 2A-B), VS treatment ameliorates aging by eliminating senescent cells in vivo (FIG. 3A-F), VS treatment shows therapeutic efficacy in bleomycin-induced lung fibrosis (FIG. 4A-D), VS treatment shows therapeutic efficacy in CC14-induced liver fibrosis (FIG. 5A-D), VS suppresses tumor growth when combined with senescence induction in A549 xenograft model (FIG. 6A-B), and VS-induced cell death can be rescued by nascent-polypeptide-associated complex (NAC) (FIG. 7A-C). Temporal mechanistic studies of VS were also conducted (FIG. 8A-B).

1. A method of inducing cell death in senescent cells comprising administering vanadyl sulfate (VS) to a subject in need thereof.

2. The method of claim 1, wherein the senescent cells are age-related senescent cells.

3. The method of claim 1, wherein the senescent cells are therapy-induced senescent cells.

4. The method of claim 1, wherein the subject suffers from senescence associated secretory phenotype (SASP).

5. The method of claim 1, wherein the subject suffers from age-related dysfunctions caused by senescence.

6. The method of claim 1, wherein the subject suffers from cardiovascular disease, a neurodegenerative disease, or a disease related to inflammation.

7. The method of claim 1, wherein the lifespan of the subject is extended.

8. A method of reducing treatment-associated side effects comprising co-administering vanadyl sulfate (VS) with a therapeutic or therapy for treating the disease.

9. The method of claim 8, wherein the therapeutic or therapy results in induction of senescence in a population of cells.

10. The method of claim 8, wherein the disease is cancer.

11. The method of claim 10, wherein the therapeutic or therapy is selected from a chemotherapeutic, radiation, and an immunotherapeutic.

12. A method of treating cancer comprising co-administering vanadyl sulfate (VS) with a therapeutic or therapy for treating cancer to a subject suffering from cancer.

13. The method of claim 12, wherein the therapeutic or therapy is selected from a chemotherapeutic, radiation, and an immunotherapeutic.
